(19) United States
(12) Patent Application Publication
(10) Pub. No.: US 2020/0391024 A1
(43) Pub. Date: Dec. 17, 2020

(54) HANDHELD NASAL STIMULATOR WITH SAFETY MECHANISM

(71) Applicant: Oculeve, Inc., Madison, NJ (US)

(72) Inventor: John L. Wardle, San Clemente, CA (US)

(21) Appl. No.: 16/896,829

(22) Filed: Jun. 9, 2020

Related U.S. Application Data
(60) Provisional application No. 62/862,551, filed on Jun. 17, 2019.

(51) Int. Cl. A61N 1/05; A61N 1/36

(52) U.S. Cl. CPC A61N 1/0546; A61N 1/36046

(57) ABSTRACT

A handheld stimulator device is described that can deliver a stimulus to tissue of a user. For example, the stimulator device can include a stimulator probe including an electrode and a deformable element configured to assist with allowing stimulus delivery by the electrode. The handheld stimulator device can also include a stimulator body configured to releasably couple to the stimulator probe. The stimulator body can include a power source and at least one contact pin positioned along the stimulator body to, during coupling of the stimulator body to the stimulator probe, form an initial contact configuration with the deformable element in an original state and cause the deformable element to transition into a deformed state. The formation of the initial contact configuration and the deformable element in the deformed state can allow delivery of a stimulus by the stimulator probe.

### CROSS-REFERENCE TO RELATED APPLICATION

The current application claims priority under 35 U.S.C. § 119(e) to U.S. Provisional patent application Ser. No. 62/862,551, filed on Jun. 17, 2019 and entitled “HANDHELD NASAL STIMULATOR WITH MECHANICALLY DEFORMING TIP DETECTION ELEMENT,” which is incorporated by reference herein in its entirety.

### TECHNICAL FIELD

The subject matter described herein relates to a handheld nasal stimulator and related methods of use.

### BACKGROUND

Dry Eye Disease (“DED”) is a condition that affects millions of people worldwide. More than 40 million people in North America have some form of dry eye, and many millions more suffer worldwide. DED results from the disruption of the natural tear film on the surface of the eye, and can result in ocular discomfort, visual disturbance, and a reduction in vision-related quality of life. Activities of daily living such as driving, computer use, housework and reading have also been shown to be negatively impacted by DED. Patients with severe cases of DED are at risk for serious ocular health deficiencies such as corneal ulceration, and can experience a quality of life deficiency comparable to that of moderate-severe angina.

The etiology of DED is becoming increasingly well understood. DED is progressive in nature, and fundamentally results from insufficient tear coverage on the surface of the eye. This poor tear coverage prevents healthy gas exchange and nutrient transport for the ocular surface, promotes cellular desiccation and creates a poor refractive surface for vision. Poor tear coverage typically results from: 1) insufficient aqueous tear production from the lacrimal glands (e.g., secondary to post-menopausal hormonal deficiency, auto-immune disease, LASIK surgery, etc.), and/or 2) excessive evaporation of aqueous tear resulting from dysfunction of the meibomian glands. Low tear volume causes a hyperosmolar environment that induces an inflamed state of the ocular surface. This inflammatory response induces apoptosis of the surface cells, which in turn prevents proper distribution of the tear film on the ocular surface so that any given tear volume is rendered less effective. This initiates a vicious cycle where more inflammation can ensue causing more surface cell damage, etc. Additionally, the neural control loop, which controls reflex tear activation, is disrupted because the sensory neurons in the surface of the eye are damaged. As a result, fewer tears are secreted and a second vicious cycle develops that results in further progression of the disease (fewer tears cause nerve cell loss, which results in fewer tears, etc.). Accordingly, effective treatment for DED is desired.

### SUMMARY

Aspects of the current subject matter can include embodiments of a handheld stimulator device including a safety mechanism for allowing and controlling use of the stimulator device, such as for stimulating facial tissue (e.g., nasal tissue) of a subject. In one aspect, the handheld stimulator device can include a stimulator probe having a nasal insertion prong including an electrode. The stimulator probe can further include a deformable element including an electrically conductive material and configured to assist with allowing stimulus delivery from the electrode of the stimulator probe. The deformable element can form an original state prior to a use of the stimulator probe and can form a deformed state after the use of the stimulator probe. The handheld stimulator device can also include a stimulator body configured to releasably couple to the stimulator probe. The stimulator body can include a power source. The stimulator body can also include at least one contact pin including an electrically conductive material and in electrical communication with the power source. The at least one contact pin can be positioned along the stimulator body to, during coupling of the stimulator body to the stimulator probe, form an initial contact configuration with the deformable element in the original state and cause the deformable element to transition into the deformed state. The formation of the initial contact configuration and the deformable element in the deformed state can allow delivery of a stimulus from the stimulator probe.

In some variations one or more of the following features can optionally be included in any feasible combination. The simulator body can include a control subsystem that, as a result of the formation of the initial contact configuration, initiates a timer defining a duration during which stimulus delivery from the stimulator probe is allowed. The duration can include a time range of approximately one hour to approximately thirty days. The control subsystem can prevent stimulus delivery after the timer has expired. The use of the stimulator probe can include coupling the stimulator probe to the stimulator body. The stimulator probe can further include at least one recess including an electrically conductive material. One or more of the at least one recess can be in electrical communication with the electrode of the nasal insertion prong. The deformable element can form, when the stimulator probe is coupled to the stimulator body, a conductive pathway that extends between at least a first contact pin and at least a first respective recess, thereby allowing the delivery of the stimulus from the electrode of the stimulator probe. The deformable element in the deformed state can prevent formation of the initial contact configuration thereby preventing initiation of a subsequent timer and reuse of the stimulator probe. The simulator probe can further include a track along which at least a part of the deformable element extends along. The track can include a securing feature for securing, when the deformable element is in the deformed state, an end of the deformable element in a position along a part of the track thereby preventing the deforming element from transitioning out of the deformed state. The end of the deformable element can form an interference fit with at least one wall of the track thereby securing the end of the deformable element in the position along the part of the track.

In another aspect, a stimulator probe for releasably coupling to a stimulator body and stimulating nasal tissue of a subject is disclosed. The stimulator probe can include a nasal insertion prong including an electrode and a deformable element including an electrically conductive material and configured to assist with allowing stimulus delivery from the electrode of the stimulator probe. The deformable element can form an original state prior to a use of the stimulator probe and forming a deformed state after the use of the stimulator probe. The original state of the deformable element can be shaped to allow formation of an initial contact configuration with at least one contact pin of the stimulator body thereby initiating a timer defining a duration during which stimulus delivery from the stimulator probe is allowed. The deformable element in the deformed state can prevent formation of the initial contact configuration thereby preventing initiation of a subsequent timer and reuse of the stimulator probe.

In some variations one or more of the following features can optionally be included in any feasible combination. The use of the stimulator probe can include coupling the stimulator probe to the stimulator body. The simulator probe can further include a track along which at least a part of the deformable element extends along. The track can include a securing feature for securing, when the deformable element is in the deformed state, an end of the deformable element in a position along a part of the track thereby preventing the deforming element from transitioning out of the deformed state. The end of the deformable element can form an interference fit with at least one wall of the track thereby securing the end of the deformable element in the position along the part of the track.

In another interrelated aspect of the current subject matter, a method of a handheld stimulator device includes coupling a stimulator probe of the handheld stimulator device to a stimulator body of the handheld stimulator device. The stimulator probe can include a nasal insertion prong including an electrode. The stimulator probe can include a deformable element including an electrically conductive material and can be configured to assist with allowing stimulus delivery from the electrode of the stimulator probe. The deformable element can form an original state prior to a use of the stimulator probe and form a deformed state after the use of the stimulator probe. The stimulator body can include a power source and at least one contact pin including an electrically conductive material and in electrical communication with the power source. The at least one contact pin can be positioned along the stimulator body to, during coupling of the stimulator body to the stimulator probe, form an initial contact configuration with the deformable element in the original state and cause the deformable element to transition into the deformed state. The formation of the initial contact configuration and the deformable element in the deformed state can allow delivery of a stimulus from the stimulator probe. The method can further include forming, as a result of the coupling, an initial contact configuration between the at least one contact pin and the deformable element in the original state. The method can further include deforming, as a result of the coupling, the deformable element into the deformed state. Additionally, the method can include allowing, as a result of the forming and the deforming, delivery of a stimulus from the electrode of the stimulator probe.

In some variations one or more of the following features can optionally be included in any feasible combination. The method can include initiating, by a control subsystem of the stimulation body and as a result of the formation of the initial contact configuration, a timer defining a duration during which stimulus delivery from the stimulator probe is allowed. The duration can include a time range of approximately one hour to approximately thirty days. The method can further include preventing, by the control subsystem, stimulus delivery from the simulator probe after the timer has expired. The stimulator probe can further include at least one recess including an electrically conductive material, and the one or more of the at least one recess can be in electrical communication with the electrode of the nasal insertion prong. The deformable element can form, when the stimulator probe is coupled to the stimulator body, a conductive pathway that extends between at least a first contact pin and at least a first respective recess, thereby allowing the delivery of the stimulus from the electrode of the stimulator probe. The deformable element in the deformed state cam prevent formation of the initial contact configuration thereby preventing initiation of a subsequent timer and reuse of the stimulator probe. The simulator probe can further include a track along which at least a part of the deformable element extends along. The track can include a securing feature for securing, when the deformable element is in the deformed state, an end of the deformable element in a position along a part of the track thereby preventing the deforming element from transitioning out of the deformed state.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIGS. 1A, 1B, 1C, 1D, and 1E show perspective, front, back, cut-away back, and cut-away side views, respectively, of an illustrative variation of a handheld stimulator;

FIG. 2 shows a block diagram schematically representing a variation of a stimulator;

FIG. 3A shows a front view of another embodiment of a handheld simulator including a safety mechanism and a stimulator probe configured to releasably couple to a stimulator body;

FIG. 3B shows a back view of the stimulator body and a bottom view of the stimulator probe of FIG. 3A, which shows a deformable element of the safety mechanism positioned along a bottom side of the stimulator probe;

FIG. 3C shows a bottom view of the stimulator probe of FIG. 3B with the deformable element in an original state;

FIG. 3D shows a close up partial view of the stimulator probe of FIG. 3B showing an elongated portion of the deformable element extending along a track and intersecting three contact features;

FIG. 3E shows a front cross-section view of the handheld stimulator of FIG. 3A showing the safety mechanism with the deformable element in an original state;

FIG. 3F shows a close up partial view of the handheld stimulator of FIG. 3E showing the deformable element in an original state and intersecting the three contact features;

FIG. 3G shows a bottom view of the stimulator probe of FIG. 3B with the deformable element in a deformed state;

FIG. 3H shows a close up partial view of the stimulator probe of FIG. 3G showing the elongated portion of the deformable element extending along the track and intersecting two out of the three contact features;

FIG. 3I shows a front cross-section view of the handheld stimulator of FIG. 3A showing the safety mechanism with the deformable element in a deformed state;

FIG. 3J shows a close up partial view of the handheld stimulator of FIG. 31 showing the deformable element in a deformed state and intersecting two out of the three contact features;

FIG. 4 shows an embodiment of the deformable element;

FIG. 5A shows another embodiment of the deformable element including a deforming coupling feature in a first position;

FIG. 5B shows the deformable element of FIG. 5A with the deforming coupling feature in a second position; and

FIG. 6 shows an embodiment of the safety mechanism including contact pins extending from the stimulator body and engaging the deformable element, thereby causing the deformable element to form the deformed state.

When practical, similar reference numbers denote similar structures, features, or elements.

### DETAILED DESCRIPTION

Described herein are stimulator devices, systems, and methods for treating one or more facial conditions (e.g., dry eye, facial and/or sinus discomfort) by providing stimulation to one or more facial features, such as facial tissue including nasal tissue, and/or sinus tissue. Generally, the stimulator devices and systems may be configured to be handheld and stimulate facial, nasal, and/or sinus tissue. In some variations, the stimulator device may include a stimulator body and a stimulator probe, where the stimulator probe includes one or more nasal insertion prongs. The stimulus delivered by the stimulator devices described herein may be electrical. For example, when the stimulator devices and systems are used to treat one or more facial conditions, the methods may include stimulating facial tissue to thereby cause an increase in tear production, reduce symptoms associated with dry eye, relieve facial and/or sinus discomfort, and/or or improve ocular appearance and/or health.

Various embodiments of a handheld stimulator device including one or more safety mechanisms are described herein. For example, some embodiments of the safety mechanism can include a control subsystem configured to authorize and limit use of a stimulator probe coupled to a stimulator body. For example, such safety mechanisms can limit a duration of use of the stimulator probe to thereby require replacement of the stimulator probe and limit or prevent biohazard exposure to a user. In some embodiments, the safety mechanism can include a deformable element that can affect formation of at least a part of a stimulation circuit configured to control delivery of a stimulus from the stimulator probe. For example, the deformable element can be part of the stimulator probe and form an original state prior to use (e.g., coupling of the stimulator probe to a stimulator body), thereby allowing the stimulator probe to deliver a stimulus. In some embodiments, the deformable element can deform as a result of coupling the stimulator probe to the stimulator body, thereby limiting use of the stimulator probe, as well as preventing re-use of the stimulator probe (e.g., recoupling the stimulator probe to the same or different stimulator body for delivering a stimulus). Various embodiments of safety mechanisms are described herein that ensure safe and effective use of various stimulator device embodiments.

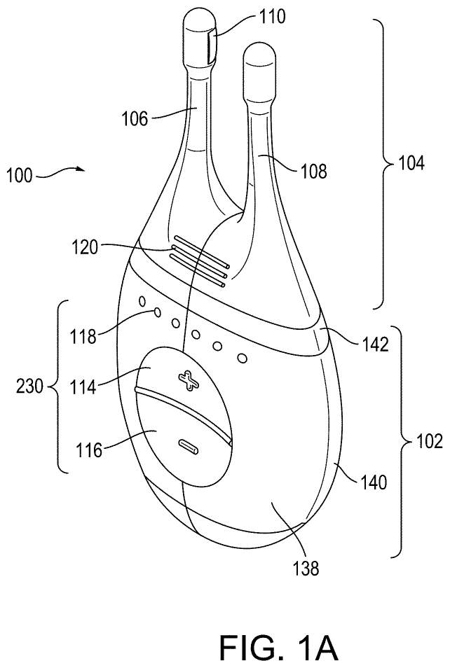

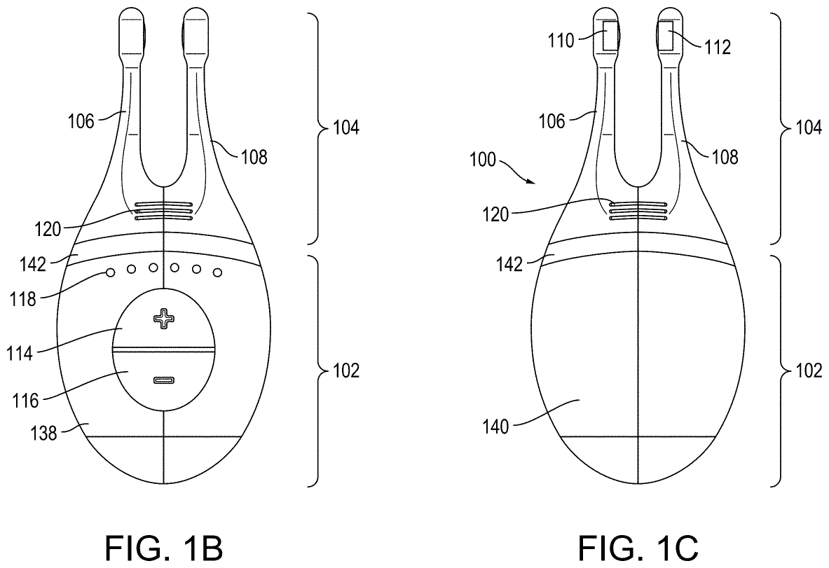

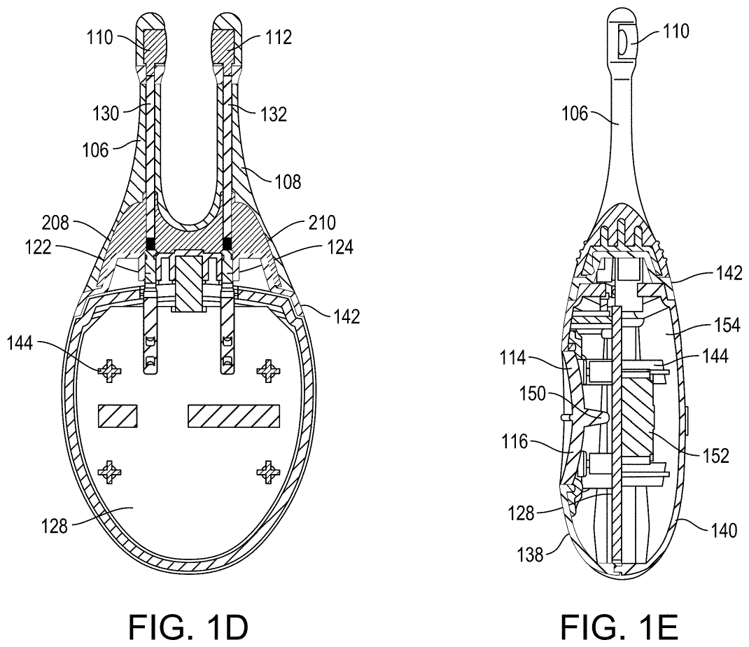

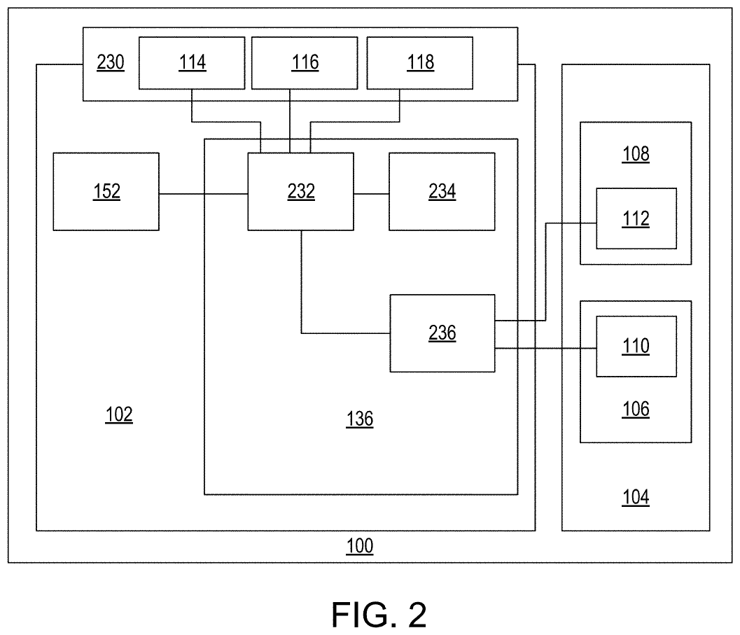

Some variations of the stimulation systems described herein may comprise a stimulator configured to be held by a user during use. FIGS. 1A, 1B, 1C, 1D, and 1E show perspective, front, back, cut-away back, and cut-away side views, respectively, of an illustrative variation of a stimulator device 100. FIG. 2 shows a block diagram schematically representing the stimulator device 100. As shown in FIGS. 1A-1E, the handheld stimulator device 100 may comprise a stimulator body 102 and a stimulator probe 104. Generally, the stimulator body 102 may be configured to generate a stimulus that may be delivered to a subject. The stimulator body 102 may contain a control subsystem 136 (as shown in FIG. 2) and a power source 152, which together may generate and control the stimulus.

The stimulus may be delivered to a subject via the stimulator probe 104. In some variations, the stimulator body 102 and stimulator probe 104 may be reversibly attachable, as described in more detail below. In other variations, the stimulator probe may be permanently connected to the stimulator body. Some or all of the stimulator 100 may be disposable. In other variations, one or more portions of the stimulator 100 may be reusable. For example, in variations where the stimulator probe 104 is releasably connected to the stimulator body 102, the stimulator body 102 may be reusable, and the stimulator probe 104 may be disposable and periodically replaced, as described in more detail below. The stimulator probe 104 may comprise at least one nasal insertion prong, which may be configured to be at least partially inserted into the nasal cavity of a subject or patient. In the handheld stimulator variation shown in FIGS. 1A-1E, the stimulator probe 104 may comprise two nasal insertion prongs 106 and 108.

In some variations, the stimulus may be electrical. In these instances, each of the two nasal insertion prongs 106 and 108 may comprise at least one electrode. As shown, the stimulator probe 104 may comprise a first electrode 110 on nasal insertion prong 106 and a second electrode 112 on nasal insertion prong 108. As shown in the cut-away view of the stimulator 100 in FIG. 1D, the first electrode 110 and second electrode 112 may be connected to leads 130 and 132 located within nasal insertion prongs 106 and 108, respectively. The leads 130 and 132 may connect directly or indirectly to the control subsystem 136 and power source 152. As such, the electrical stimulus may travel from the control subsystem 136, through the leads 130 and 132, and through the electrodes 110 and 112.

The stimulator body 102 may comprise a user interface 230 comprising one or more operating mechanisms to adjust one or more parameters of the stimulus. The operating mechanisms may provide information to the control subsystem 136, which may comprise a processor 232, memory 234, and/or stimulation subsystem 236, as shown in FIG. 2. In some variations, the operating mechanisms may comprise a first button 114 and a second button 116. In some variations, pressing the first button 114 may turn on the stimulator 100 and/or change one or more parameters of the stimulus (e.g., increase the intensity of the stimulus, change the stimulation pattern, or the like), while pressing the second button 116 may turn off the stimulator 100 and/or change one or more parameters of the stimulus (e.g., decrease the intensity of the stimulus, change the stimulation pattern, or the like). Additionally or alternatively, the user interface may comprise one or more feedback elements (e.g., based on light, sound, vibration, or the like). As shown, the user feedback elements may comprise light-based indicators 118, which may provide information to the user. Additionally or alternatively, in some variations the stimulator body may comprise a display, which may be configured to convey information to a user via text and/or images. Additionally or alternatively, the stimulator body may comprise a speaker or buzzer configured to produce one or more speech prompts or other sounds. Additionally or alternatively, the stimulator body may be configured to vibrate, such as via a vibration motor (e.g., mounted on a printed circuit board assembly including the power source 152).

As discussed above, the stimulator 100 may comprise a power source 152. The power source may be any suitable power supply capable of powering one or more functions of the stimulator, such as one or more batteries, capacitors, or the like. While the stimulator body 102 comprises a power source 152, in other variations the stimulator body need not comprise a power source. In some variations, the stimulator body may comprise a port, cord, or other mechanism for connecting the stimulator to an external power source (such as a wall outlet or separate battery pack), which in turn may be used to power one or more portions of the stimulator.

Generally, the processor 232 may be configured to control operation of the various subsystems of the control subsystem 136. For example, the processor 232 may be configured to control the stimulation subsystem 236 to control parameters of the stimulation provided by the stimulation subsystem 236. The memory 234 may be configured to store programming instructions for the stimulator, and the processor 232 may use these programming instructions in controlling operation of the stimulator device 100. The stimulation subsystem 236 may be configured to generate a stimulation signal and deliver the stimulation signal to a patient via the stimulator probe 104.

Additionally or alternatively, the control subsystem 136 may comprise a communications subsystem. The communication subsystem may be configured to facilitate communication of data and/or energy between the stimulator device 100 and an external source.

In some embodiments, the control subsystem 136 may include and/or be a part of one or more safety mechanisms, such as a safety mechanism that controls and/or limits a duration of use of a stimulator probe 104 to thereby require replacement of the stimulator probe 104 and limit or prevent biohazard exposure to a user. For example, the processor 232 may comprise software that assists with authorizing use and/or limiting the duration of use of a stimulator probe 104.

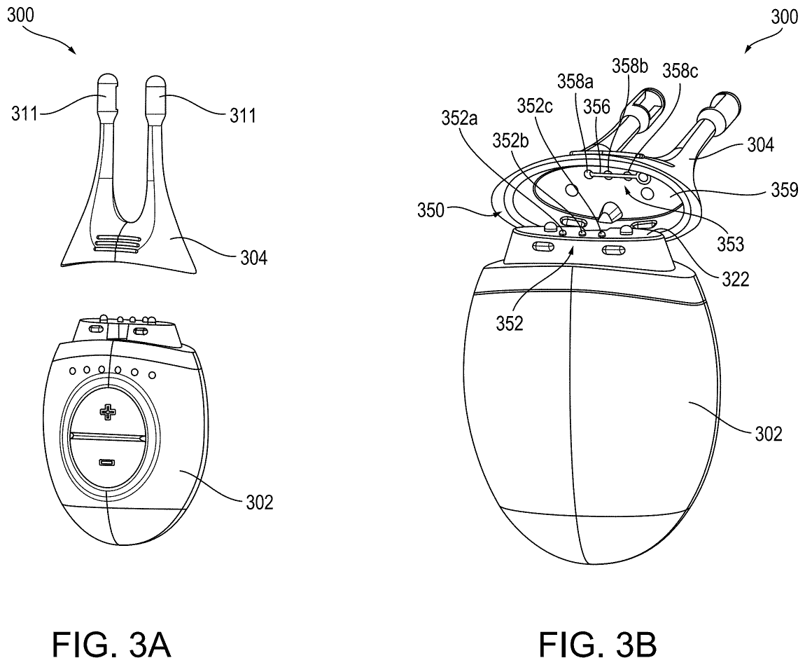

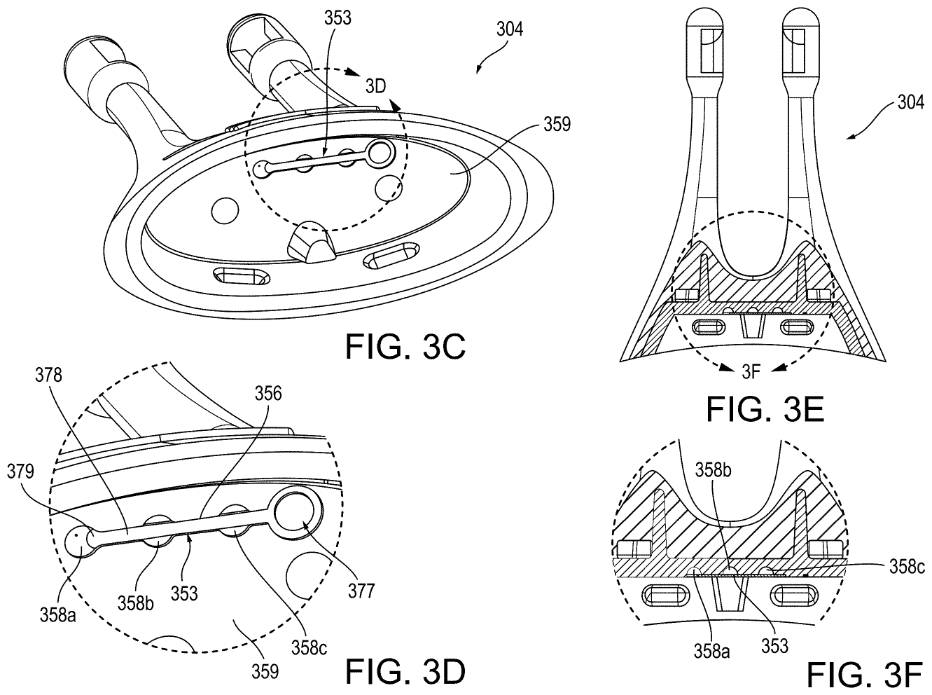

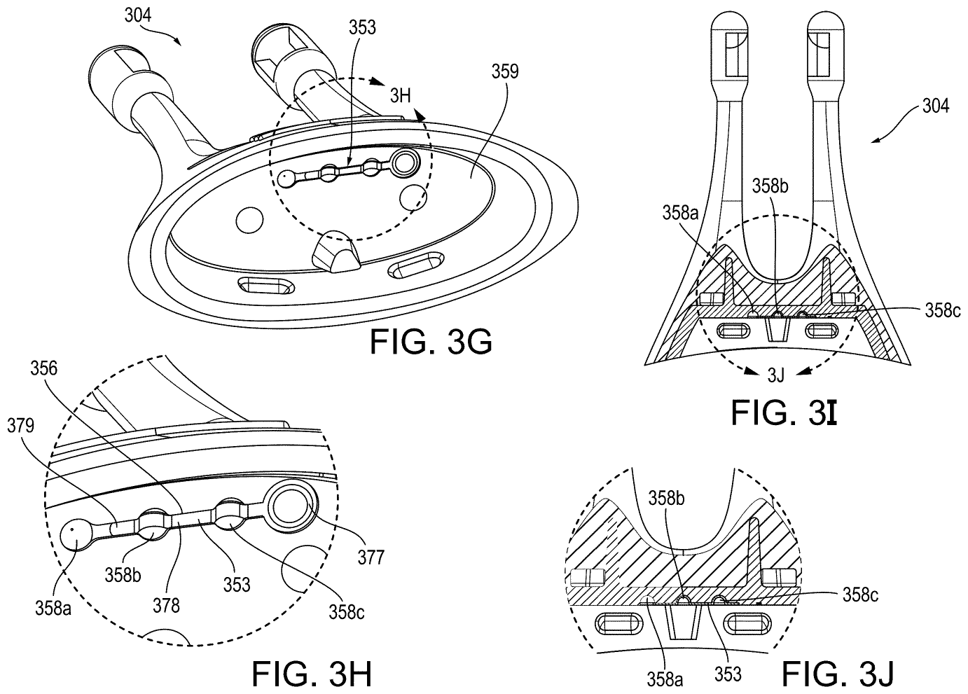

In some embodiments, the control subsystem 136 may prevent delivery of a stimulus (e.g., prevent current between power source 152 and electrodes 110, 112) when the safety mechanism described with respect to FIGS. 3A-3F detects use of a stimulator probe 104 that exceeds a predefined threshold. For example, the control subsystem 136 may prevent delivery of a stimulus after detecting the stimulator probe 104 has been coupled to a stimulator body 102 longer than a predefined duration. As such, a user would have to replace the stimulator probe 104 in order for the control subsystem 136 to allow delivery of a stimulus, such as with a new stimulator probe 104 coupled to the stimulator body 102. In some variations, the predefined duration may be limited to approximately one hour to approximately 30 days, however, other predefined durations are within the scope of this disclosure. Other safety mechanisms may be included in a stimulator embodiment and may be in communication with the control subsystem 136 for ensuring safe and effective use of the stimulator device 100, as will be described in greater detail below.

As discussed above, some embodiments of the handheld stimulator 100 include a reusable stimulator body 102 and a disposable stimulator probe 104. In such an embodiment, the stimulator probe 104 may releasably couple to the stimulator body 102. For example, the stimulator body 102 may be configured to include the power generator (e.g., power source 152) that produces an electrical stimulation waveform and the stimulator probe 104 may be configured to be inserted in a nasal cavity of a user to deliver the neurostimulation therapy. During use, the disposable stimulator probe 104 can contact nasal mucosa, nasal fluid, etc. As such, the stimulator probe 104 may be unsanitary after a single use. Due to the small size and complex geometry of the stimulator probe 104, it may be inefficient and/or ineffective for a user to sanitize the stimulator probe 104 after use. As such, routine replacement of the stimulator probe 104 may be necessary to eliminate risks of biohazard exposure to the user. As such, various safety mechanisms are described herein for controlling and limiting use of stimulator probes 104, thereby requiring replacement of stimulator probes, such as after a single use and/or preventing use of stimulator probe 104 that has previously been coupled to a stimulator body 102.

For example, some safety mechanisms of the handheld stimulator device 100 include a deformable element coupled to a coupling interface of a stimulator probe. The deformable element can be configured to assist with allowing and/or limiting use of the associated stimulator probe, as described in detail below. For example, the deformable element can assist with forming at least a part of a stimulation circuit for allowing stimulation to be delivered by the associated stimulator probe. Additionally, the deformable element can prevent or disrupt formation of at least a part of the stimulation circuit, thereby preventing use or limiting use (e.g., based on a predefined time) of the associated stimulator probe. Various safety mechanism embodiments, including various deformable elements, of handheld stimulators 100 are described in greater detail below.

FIGS. 3A-3J illustrate another embodiment of a handheld simulator device 300 including a stimulator probe 304 configured to releasably couple to a stimulator body 302 and includes any one or more features described above with respect to the stimulator probe 104 and stimulator body 102 described above with respect to FIGS. 1A-2. The handheld stimulator device 300 illustrated in FIGS. 3A-3J also includes a safety mechanism 350 configured to control and limit use of the stimulator probe 304.

For example, the safety mechanism 350 can include a stimulation circuit that can be formed as a result of a stimulator probe 304 being coupled to a stimulator body 302. The stimulation circuit can provide electrical communication between a power generator (e.g., power source 152 of FIG. 2) of the stimulator body 302 and at least one electrode 311 of the stimulator probe 304, such as for allowing stimulus delivery via one or more electrodes 311. The safety mechanism 350 and stimulation circuit can include a control subsystem (e.g., control subsystem 136 of FIG. 2) configured to control delivery of the stimulus, such as limit and/or prevent stimulus delivery, as will be described in greater detail below.

As shown in FIG. 3C, the safety mechanism 350 can include a deformable element 353 coupled to a coupling interface 359 of the stimulator probe 304. The deformable element 353 can be made out of a conductive material and assist with forming the stimulation circuit. Additionally, the deformable element 353 can form more than one shape or state that allows and/or prevents the flow of current along one or more parts of the stimulation circuit. For example, a new, unused stimulator probe 304 can include a deformable element 353 that is in an original state (e.g., not deformed) and thus is configured to allow formation of a first conductive pathway as a result of the stimulator probe 304 being coupled to the stimulator body 302. For example, the control subsystem of the stimulation circuit can initiate a timer as a result of the formation of the first conductive pathway, which can be a part of the stimulation circuit. The timer can define a predefined duration of allowed stimulus delivery of the stimulator probe 304. As such, once the timer expires (e.g., reaches or runs out a predefined time), the stimulator probe 304 may be deactivated (e.g. prevented from delivering stimulation). As such, after the timer has expired, the control subsystem can prevent stimulus delivery with the associated stimulator probe 304, thus requiring replacement with a new stimulator probe 304 in order to continue stimulus delivery. In addition, as a result of the stimulator probe 304 being coupled to the stimulator body 302, the deformable element 353 can form a deformed state that prevents formation of the first conductive pathway and thus limits and/or prevents use of the associated stimulator probe 304, as will be described in greater detail below. Such deactivation of the stimulator probe 304 by the safety mechanism 350 can prevent against prolonged use of the stimulator probe 304 and require a user to replace the stimulator probe, thereby at least reducing biohazard exposure to the user.

The deformable element 353 can include a variety of shapes and sizes. In some embodiments, as shown in FIG. 3D, the deformable element 353 can include a first end 377 configured to be coupled to the coupling interface 359 of the stimulator probe 304. The deformable element 353 can include an elongated portion 378 that extends between the first end 377 and an opposing second end 379. The second end 379 and at least a part of the elongated portion 378 of the deformable element 353 can be in contact with the coupling interface 359 of the stimulator probe 304 but not secured to the coupling interface 359 and thus free to move relative to the coupling interface 359, which can assist with allowing the deformable element 353 to form more than one shape or state (e.g., original state and deformed state), as further described below. Other shapes and configurations of the deformable element 353 are within the scope of this disclosure. For example, the deformable element 353 can be made out of one or more materials, such as annealed 316 stainless steel that can readily deform and resist stretching (e.g., prevent lengthening).

As shown in FIGS. 3A-3E, the stimulator body 302 can include at least one contact pin 352 that is electrically conductive and in electrical communication with a power generator (e.g., power source 152 of the stimulator body). As shown in FIG. 3B, the stimulator probe 304 may include the deformable element 353 along the coupling interface 359 of the stimulator probe 304, and the stimulator body 302 may include the at least one contact pin 352 along a complimenting coupling interface 322 of the stimulator body 302. For example, the coupling interfaces 359 and 322 of the stimulator probe 304 and stimulator body 302, respectively, can assist with aligning and securing a coupling therebetween, including aligning and allowing an engagement between at least one contact pin 352 and the deformable element 353 as a result of the stimulator probe 304 being coupled to the stimulator body 302. As will be described in greater detail below, the deformable element 353 may be configured to change shape or deform after or upon coupling of the stimulator probe 304 to the stimulator body 302. For example, during coupling of the stimulator probe 304 to the stimulator body 302, at least one contact pin 352 can contact and apply a force against a side of the deformable element 353, thereby causing the deformable element 353 to deform (e.g., transition from an original state to a deformed state).

As shown in FIGS. 3A-3E, in some embodiments the stimulator body 302 can include three contact pins 352 (352a, 352b, 352c) that can be made from one or more of a variety of conductive material (e.g., 316 stainless steel). In some embodiments, the deformable element 353 can extend along a track 356 along the coupling interface 359 of the stimulator probe. The track 356 can include an elongated recess and/or intersect three contact features 358. For example, each of the contact features 358 can include a recessed rounded or spherical shape and can be at least partly made out of an electrically conductive material. For example, the contact feature 358 can be configured to allow at least a part of a respective contact pin 352 (e.g., having a rounded end) to extend therein, such as when the stimulator probe 304 is coupled to the stimulator body 302. As such, the deformable element 353 positioned between each contact pin 352 and respective contact feature 358 can become deformed when the stimulator probe 304 is coupled to the stimulator body 302. For example, the contact pin 352 can push a part of the deformable element 353 into the respective contact feature 358, which can result in the deformable element 353 having a shorter overall length and extend a shorter distance along the track 356, as well as not intersecting all of the contact features 358, as shown in FIG. 3H. Furthermore, such deforming of the deformable element 353 can form an electrically conductive pathway between the contact pin 352 and the respective contact feature 358 thereby forming a part of the stimulation circuit, such as for allowing delivery of a stimulus.

In some embodiments, the deformable element 353 can include a flat, rectangular shaped elongated portion 378 and a spherical or rounded second end 379, as shown in FIG. 3D. As discussed above, the first end 377 can be secured in position along the stimulator probe 304 and the second end 379 can be allowed to move. As such, when the stimulator probe 304 is coupled to the stimulator body 302, the deformable element 353 can be deformed (e.g., into the deformed state) thereby causing the second end 379 of the deformable element 353 to move along the track 356, such as towards the first end 377, such as shown in FIG. 3H. As shown in FIGS. 3D, the rounded second end 379, for example, can include a width that is greater than a width of the elongated portion 378 of the deformable element 353.

In some embodiments, the track 356 can include a securing feature for securing the second end 379 of the deformable element 353 in a position along the track 356, such as for preventing the deformable element 353 from becoming disengaged from the track 356 and/or transitioning back to the original state. For example, some embodiments of the track 356 can include an elongated recess including opposing track sides spaced away from each other a distance that is approximately the same as or slightly greater than the width of the elongated portion 378 thereby allowing at least a part of the elongated portion 378 to slideably engage with the opposing sides of the track. Additionally, at least one of the opposing track sides can include a compressible material (e.g., made out of a polymer, such as ABS). Furthermore, the distance between the opposed sides of the track 356 can also be less than the width of the second end 379 of the deformable element 353, thereby forming an interference fit between at least one of the opposed sides of the track 356 and the second end 379 of the deformable element 353. As such, when the deformable element 353 forms the deformed state and includes a shorter overall length, the second end 379 can be positioned along the track 356 for forming an interference fit with the track 356, as shown, for example, in FIG. 3H. Such interference fit can prevent movement of the second end 379 thereby preventing the deformable element 353 from becoming disengaged from the track 356 and/or transitioning out of the deformed state. This can assist with preventing tampering of the safety mechanism 350, such as a user attempting to re-activate (e.g., activate a subsequent timer) and reuse the stimulator probe 304.

As shown in FIG. 3D, in the original, un-deformed state, the deformable element 353 can intersect all three contact features 358 (as shown in FIG. 3D), and in the deformed state, the deformable element 353 can intersect only the second and third contact features 358b and 358c, respectively. Such deformation of the deformable element 353 can assist in limiting use of the stimulator probe 304, as will be discussed further below.

The contact pins 352 can be in electrical communication with the power source and can be a part of the stimulation circuit that allows delivery of a stimulus. In addition, the stimulation circuit can include at least two electrical pathways that are each in communication with the control subsystem (e.g., control subsystem 136) that is configured to assist with monitoring and controlling use of the stimulator probe 304. For example, a first electrical pathway can allow and monitor current between the first contact pin 352a and the third contact pin 352c, and a second electrical pathway can at least monitor current between the second contact pin 352b and the third contact pin 352c. The deformable element 353 is at least partly made out of a conductive material, which can allow current to flow between contact pins 352 that are in contact with the deformable element 353 and/or between contact pins 352 and contact features 358 that are in contact with the deformable element 353.

For example, when coupling a new, unused stimulator probe 304 to a stimulator body 302, an initial contact configuration can be formed between the contact pins 352 and the deformable element 353. For example, all three contact pins 352 can contact the deformable element 353 that is in an original, flat configuration and that intersects all three contact features 358a, 358b, 358c. When a signal from the first contact pin 352a and third contact pin 352c are recognized by the control subsystem (e.g., as a result of a first electrical pathway formation), an internal clock associated with the power generator of the stimulator body 302 can be activated by the control subsystem. Such activation can include starting a predefined or programmed timer that defines a service life of the stimulator probe 304 (e.g., 1 hour, 6 hours, 12 hours, 24 hours, 48 hours, 30 days, etc.). As such, after activation of the timer and before the timer expires, the stimulator probe 304 can be allowed to deliver at least one stimulus. Furthermore, when the predefined timer ends, the stimulator body 302 and/or stimulator probe 304 may be deactivated and/or unable to be used, thereby requiring the user to replace the current stimulator probe 304 with a new stimulator probe 304.

In some embodiments, after the control subsystem detects a current along the first electrical pathway and the second electrical pathway, the control subsystem of the stimulator body 302 can recognize that an acceptable stimulator probe 304 is coupled to the stimulator body 302 and allow stimulus delivery from the electrodes 311 of the stimulator probe 304.

Furthermore, to ensure the stimulator probe 304 is not re-used, if a user re-couples the stimulator probe 304 including a deformable element 353 in a deformed state to a stimulator body 302, the first electrical pathway can be unable to form. For example, the deformed deformable element 353 can no longer reach the first contact feature 358a and/or first contact pin 352a, thereby preventing formation of the first electrical pathway. Formation of the first electrical pathway can activate the internal clock, which can allow power to be delivered to the stimulator probe 304 for delivering stimulation. As such, the safety mechanism 350 can assist with preventing reuse or overuse of a stimulator probe 304, which can limit or prevent biohazard exposure to a user.

Other embodiments of the safety mechanism are within the scope of this disclosure. For example, the deformable element can include a variety of shapes and features, some of which are described in greater detail below.

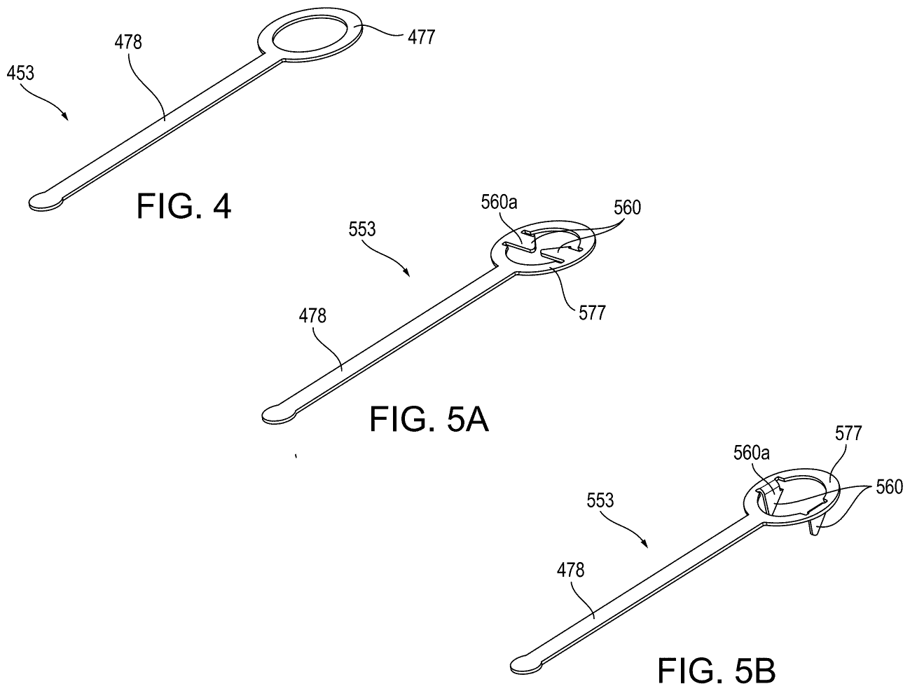

FIG. 4 shows an embodiment of the deformable element 453 including a first end 477 and an elongated portion 478 extending from the first end 477. The first end 477 can include a ring shape to allow a center of the ring to mate with a feature along the stimulator probe 304, such as for securely positioning at least a part of the deformable element 453 along the coupling interface of the stimulator probe. In some embodiments, the ring shaped first end 477 of the deformable element 453 can be coupled to a feature that can deform (e.g., melt, bend, heat stake, etc.) to thereby secure at least the first end 477 of the deformable element 453 to the stimulator probe. As such, the deformable element 453 can deform, such as long the elongated portion 478 and cause the second end 479 to move closer to the first end 477 to form the deformed state.

FIGS. 5A and 5B illustrate another embodiment of the deformable element 553 including a first end 577 and an elongated portion 578 extending from the first end 577. As shown in FIG. 5A, the ring shaped first end 577 can include a deforming coupling feature 560 including at least one barbed feature 560a that can assist with securing the first end 577 of the deformable element 553 to the stimulator probe. For example, the deforming coupling feature 560 can form a first position, as shown in FIG. 5A, and a second position, as shown in FIG. 5B. In some embodiments, the at least one barbed feature of the deforming coupling feature 560 can be forced into one or more holes or cavities that are smaller than the coupling features 560 in the second, deformed position to thereby mechanically secure the deformable element 553 to the stimulator probe 304.

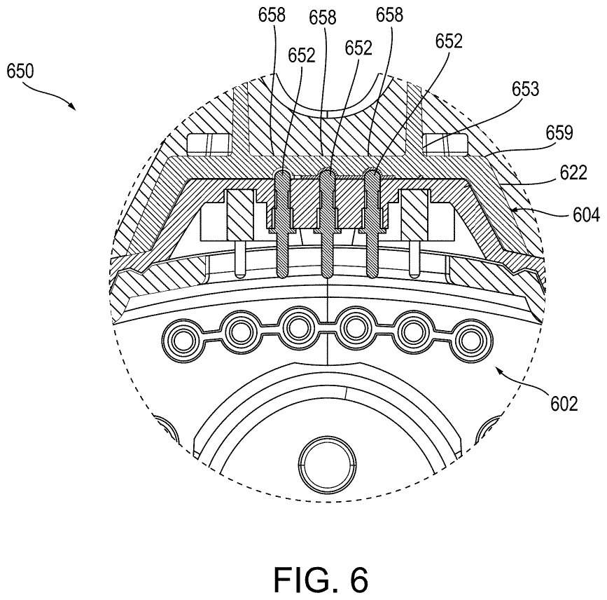

FIG. 6 shows an embodiment of the safety mechanism 650 including contact pins 652 extending from a coupling interface 622 of the stimulator body 602 for engaging the deformable element 653 and/or contact features 658 along a complimenting coupling interface 659 of the stimulator probe 604. As shown in FIG. 6, distal ends of the contact pins 652 can include a rounded end and the contact features 658 can each include a complimentary rounded recess. For example, the contact feature 658 can be sized and shaped to allow the rounded end of the contact pin 652 to extend into the recessed contact feature 658. Additionally, as shown in FIG. 6, the contact features 658 can include a rounded and/or spherical shape, as well as shaped to allow the deformable element 653 to also extend into the recess between the rounded end of the contact pin 652 and the contact feature 658. The deformable element 653 can thus provide an electrically conductive pathway between the contact pin 652 and the contact features 658. Such electrically conductive pathway can allow stimulus delivery, such as an electrical stimulus from the power source that is delivered via one or more electrodes of the stimulator probe. As shown in FIG. 6, the deformed deformable element 653 may not extend to at least one contact feature 658, such as between a first contact pin 652a and a first contact feature 658a. As such, a gap or break in the stimulation circuit can be formed thereby preventing a detection of a current along the part of the stimulation circuit and thus limiting and/or preventing stimulus delivery by the stimulator device, as discussed above.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. A handheld stimulator device for stimulating nasal tissue of a subject, comprising:
a stimulator probe, wherein the stimulator probe comprises:
a nasal insertion prong comprising an electrode;
a deformable element comprising an electrically conductive material and configured to assist with allowing stimulus delivery from the electrode of the stimulator probe, the deformable element forming an original state prior to a use of the stimulator probe and forming a deformed state after the use of the stimulator probe; and
a stimulator body configured to releasably couple to the stimulator probe, wherein the stimulator body comprises:
a power source; and
at least one contact pin comprising an electrically conductive material and in electrical communication with the power source, the at least one contact pin positioned along the stimulator body to, during coupling of the stimulator body to the stimulator probe, form an initial contact configuration with the deformable element in the original state and cause the deformable element to transition into the deformed state, the formation of the initial contact configuration and the deformable element in the deformed state allowing delivery of a stimulus from the stimulator probe.

2. The handheld stimulator device of claim 1, wherein the simulator body comprises a control subsystem that, as a result of the formation of the initial contact configuration, initiates a timer defining a duration during which stimulus delivery from the stimulator probe is allowed.

3. The handheld stimulator device of claim 2, wherein the duration includes a time range of approximately one hour to approximately thirty days.

4. The handheld stimulator device of claim 3, wherein the control subsystem prevents stimulus delivery after the timer has expired.

5. The handheld stimulator device of claim 1, wherein the use of the stimulator probe comprises coupling the stimulator probe to the stimulator body.

6. The handheld stimulator device of claim 1, wherein the stimulator probe further comprises:
at least one recess comprising an electrically conductive material, one or more of the at least one recess being in electrical communication with the electrode of the nasal insertion prong,
wherein the deformable element forms, when the stimulator probe is coupled to the stimulator body, an electrically conductive pathway that extends between at least a first contact pin and at least a first respective recess, thereby allowing the delivery of the stimulus from the electrode of the stimulator probe.

7. The handheld stimulator device of claim 2, wherein the deformable element in the deformed state prevents formation of the initial contact configuration thereby preventing initiation of a subsequent timer and reuse of the stimulator probe.

8. The handheld stimulator device of claim 7, wherein the simulator probe further comprises a track along which at least a part of the deformable element extends along, the track including a securing feature for securing, when the deformable element is in the deformed state, an end of the deformable element in a position along a part of the track thereby preventing the deforming element from transitioning out of the deformed state.

9. The handheld stimulator device of claim 8, wherein the end of the deformable element forms an interference fit with at least one wall of the track thereby securing the end of the deformable element in the position along the part of the track.

10. A stimulator probe for releasably coupling to a stimulator body and stimulating nasal tissue of a subject, the stimulator probe comprising:
a nasal insertion prong comprising an electrode;
a deformable element comprising an electrically conductive material and configured to assist with allowing stimulus delivery from the electrode of the stimulator probe, the deformable element forming an original state prior to a use of the stimulator probe and forming a deformed state after the use of the stimulator probe, wherein the original state of the deformable element is shaped to allow formation of an initial contact configuration with at least one contact pin of the stimulator body thereby initiating a timer defining a duration during which stimulus delivery from the stimulator probe is allowed, and
wherein the deformable element in the deformed state prevents formation of the initial contact configuration thereby preventing initiation of a subsequent timer and reuse of the stimulator probe.

11. The stimulator probe of claim 10, wherein the use of the stimulator probe comprises coupling the stimulator probe to the stimulator body.

12. The stimulator probe of claim 10, wherein the simulator probe further comprises a track along which at least a part of the deformable element extends along, the track including a securing feature for securing, when the deformable element is in the deformed state, an end of the deformable element in a position along a part of the track thereby preventing the deforming element from transitioning out of the deformed state.

13. The stimulator probe of claim 12, wherein the end of the deformable element forms an interference fit with at least one wall of the track thereby securing the end of the deformable element in the position along the part of the track.

14. A method of a handheld stimulator device, the method comprising:
coupling a stimulator probe of the handheld stimulator device to a stimulator body of the handheld stimulator device, the stimulator probe comprising:
a nasal insertion prong comprising an electrode;
a deformable element comprising an electrically conductive material and configured to assist with allowing stimulus delivery from the electrode of the stimulator probe, the deformable element forming an original state prior to a use of the stimulator probe and forming a deformed state after the use of the stimulator probe; and
the stimulator body comprising:
a power source; and
at least one contact pin comprising an electrically conductive material and in electrical communication with the power source, the at least one contact pin positioned along the stimulator body to, during coupling of the stimulator body to the stimulator probe, form an initial contact configuration with the deformable element in the original state and cause the deformable element to transition into the deformed state, the formation of the initial contact configuration and the deformable element in the deformed state allowing delivery of a stimulus from the stimulator probe;
forming, as a result of the coupling, an initial contact configuration between the at least one contact pin and the deformable element in the original state;
deforming, as a result of the coupling, the deformable element into the deformed state; and
allowing, as a result of the forming and the deforming, delivery of a stimulus from the electrode of the stimulator probe.

15. The method of claim 14, further comprising:
initiating, by a control subsystem of the stimulation body and as a result of the formation of the initial contact configuration, a timer defining a duration during which stimulus delivery from the stimulator probe is allowed.

16. The method of claim 15, wherein the duration includes a time range of approximately one hour to approximately thirty days.

17. The method of claim 14, further comprising:
preventing, by the control subsystem, stimulus delivery from the simulator probe after the timer has expired.

18. The method of claim 14, wherein the stimulator probe further comprises:
at least one recess comprising an electrically conductive material, one or more of the at least one recess being in electrical communication with the electrode of the nasal insertion prong,
wherein the deformable element forms, when the stimulator probe is coupled to the stimulator body, a conductive pathway that extends between at least a first contact pin and at least a first respective recess, thereby allowing the delivery of the stimulus from the electrode of the stimulator probe.

19. The method of claim 15, wherein the deformable element in the deformed state prevents formation of the initial contact configuration thereby preventing initiation of a subsequent timer and reuse of the stimulator probe.

20. The method of claim 19, wherein the simulator probe further comprises a track along which at least a part of the deformable element extends along, the track including a securing feature for securing, when the deformable element is in the deformed state, an end of the deformable element in a position along a part of the track thereby preventing the deforming element from transitioning out of the deformed state.
